(19) United States
(12) Patent Application Publication
(10) Pub. No.: US 2014/0243616 A1
(43) Pub. Date: Aug. 28, 2014

(54) SYSTEMS AND METHODS FOR DETECTING CARDIOVASCULAR DISEASE

(71) Applicant: AUM Cardiovascular, Inc., Northfield, MN (US)

(72) Inventor: Marie A. Johnson, NORTHFIELD, MN (US)

(21) Appl. No.: 14/270,541

(22) Filed: May 6, 2014

Related U.S. Application Data
(63) Continuation of application No. 12/962,812, filed on Dec. 8, 2010.
(60) Provisional application No. 61/267,803, filed on Dec. 8, 2009.
(60) Provisional application No. 61/406,422, filed on Oct. 25, 2010.

(51) Int. Cl. A61B 7/04; A61B 5/0205; A61B 5/03; A61B 5/00; A61B 5/02

(52) U.S. Cl. CPC A61B 7/045; A61B 5/6823; A61B 5/6822; A61B 5/6824; A61B 5/6826; A61B 5/02028; A61B 5/6831; A61B 5/7282; A61B 5/742; A61B 5/0004; A61B 5/03; A61B 5/4833; A61B 5/7475; A61B 5/0205
USPC 600/301; 600/586; 600/481; 600/300; 600/561

(57) ABSTRACT

Embodiments of the invention provide such an improved system and method. In particular, embodiments relate to systems, methods and apparatuses which use acoustic data in the detection of coronary artery disease. Embodiments can enable fast, non-invasive identification of clinically relevant coronary artery disease, which can ultimately save lives. The non-invasive nature is one example of a multitude of convenient aspects of embodiments that can be used to meet a large, as yet unmet need in a cost-effective and accurate manner. Results can be provided in real-time and with clarity, providing quick and easily understandable indications that can shorten the path to intervention for patients, making embodiments suitable for a wide range of environments, purposes, users and patients.

### RELATED APPLICATION

This application is a continuation of application Ser. No. 12/962,812 filed Dec. 8, 2010, which claims the benefit of U.S. Provisional Application Nos. 61/267,803 filed Dec. 8, 2009, and 61/406,422 filed Oct. 25, 2010, each of which is hereby fully incorporated herein by reference.

### TECHNICAL FIELD

This invention relates generally to the medical diagnostics field, and more specifically to improved systems and methods for detecting cardiovascular disease in the medical diagnostics field.

### BACKGROUND

Cardiovascular disease affects the lives of millions of people, and may affect the health of a patient without warning. In particular, detection of coronary artery stenosis (occlusion of the coronary arteries) typically involves evaluating patient history, performing a physical examination, stress testing, and/or performing a coronary angiogram. An evaluation of patient history and performing a physical examination, however, may not provide enough information for a confident conclusion, and although stress testing is frequently ordered to detect possible coronary artery disease, the sensitivity and specificity of the stress test varies greatly, depending on whether there is single or multi-vessel disease. Furthermore, a coronary angiogram is an invasive procedure that may carry significant cost and/or risk to the patient.

Thus, there is a need in the medical diagnostics field to create an improved system and method for detecting coronary artery disease.

### SUMMARY

Embodiments of the invention provide such improved systems and methods. In particular, embodiments relate to systems, methods and apparatuses which use acoustic data in the detection of coronary artery disease. Embodiments can enable fast, non-invasive identification of clinically relevant coronary artery disease, which can ultimately save lives. The non-invasive nature is one example of a multitude of convenient aspects of embodiments that can be used to meet a large, as yet unmet need in a cost-effective and accurate manner. Results can be provided in real-time and with clarity, providing quick and easily understandable indications that can shorten the path to intervention for patients, making embodiments suitable for a wide range of environments, purposes, users and patients.

In an embodiment, a medical device comprises at least one acoustic sensor; a housing coupled to the at least one acoustic sensor and configured to position the at least one acoustic sensor to collect acoustic data, the acoustic data including a representation of at least a sound of blood through an artery of a user; a processor operably coupled to the acoustic sensor and configured to receive the acoustic data and determine a health-related event based at least on the acoustic data; and a securing mechanism configured to secure the housing to a portion of the user.

In another embodiment, a device for use in the detection of a health-related event, the device comprises a main body; a sensor operably coupled to the main body; an electronic subsystem housed substantially within the main body and configured to condition data received from the sensor; a processor coupled to the electronic subsystem and configured to receive the conditioned data and determine a health-related event; an electronic display operably coupled to the main body and configured to visually display at least one of the health-related event or the conditioned data; a battery operably coupled to the main body and configured to provide power to the sensor, the electronic subsystem, the processor, and the electronic display; and a band configured to secure the main body to an arm of a user.

In another embodiment, a system for determining a health-related event comprises a device comprising at least one sensor, a housing coupled to the at least one sensor and configured to position the at least one sensor to collect sensor data, the sensor data including a representation of at least a sound of blood through an artery of a user, a processor operably coupled to the sensor and configured to receive the sensor data, and a securing mechanism configured to secure the housing to a body of a user; and a remote electronic device in communication with the processor, wherein the processor is configured to transmit the sensor data to the remote electronic device, and the remote electronic device is configured to determine a health-related event based at least on the sensor data.

### BRIEF DESCRIPTION OF THE DRAWINGS

The invention may be more completely understood in consideration of the following detailed description of various embodiments of the invention in connection with the accompanying drawings, in which:

FIG. 1 depicts a system for detecting cardiovascular disease according to an embodiment.

FIG. 2 depicts a sensor housing according to an embodiment.

FIG. 3A depicts a sensor housing according to an embodiment.

FIG. 3B depicts a sensor housing according to an embodiment.

FIG. 3C depicts a sensor housing according to an embodiment.

FIG. 4 depicts a sensor housing according to an embodiment.

FIG. 5A depicts a sensor housing according to an embodiment.

FIG. 5B depicts another view of the sensor housing of FIG. 5A.

FIG. 6 depicts a system according to an embodiment.

FIG. 7 depicts a system according to an embodiment.

FIG. 8 depicts a system according to an embodiment.

FIG. 9 depicts a system according to an embodiment.

FIG. 10 depicts a system according to an embodiment.

FIG. 11A depicts a system according to an embodiment.

FIG. 11B depicts a system according to an embodiment.

FIG. 12A depicts a graphical user interface presented on a display according to an embodiment.

FIG. 12B depicts a graphical user interface presented on a display according to an embodiment.

FIG. 12C depicts a graphical user interface presented on a display according to an embodiment.

FIG. 13A depicts a graphical user interface according to an embodiment.

FIG. 13B depicts a graphical user interface according to an embodiment.

FIG. 14A depicts a graphical user interface according to an embodiment.

FIG. 14B depicts a graphical user interface according to an embodiment.

FIG. 15A depicts a graphical user interface according to an embodiment.

FIG. 15B depicts a graphical user interface according to an embodiment.

FIG. 16A depicts a graphical user interface according to an embodiment.

FIG. 16B depicts a graphical user interface according to an embodiment.

FIG. 16C depicts a graphical user interface according to an embodiment.

FIG. 16D depicts a graphical user interface according to an embodiment.

FIG. 17 depicts a system according to an embodiment.

FIG. 18 depicts a system according to an embodiment.

FIG. 19 is a flowchart of a method according to an embodiment.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

Embodiments of the invention relate to systems and methods for detecting cardiovascular disease. Advantages of embodiments provide fast, non-invasive systems and methods for the identification of clinically relevant coronary artery disease. The following description of embodiments of the invention is not intended to limit the invention to these embodiments, but rather to enable any person skilled in the art to make and use this invention.

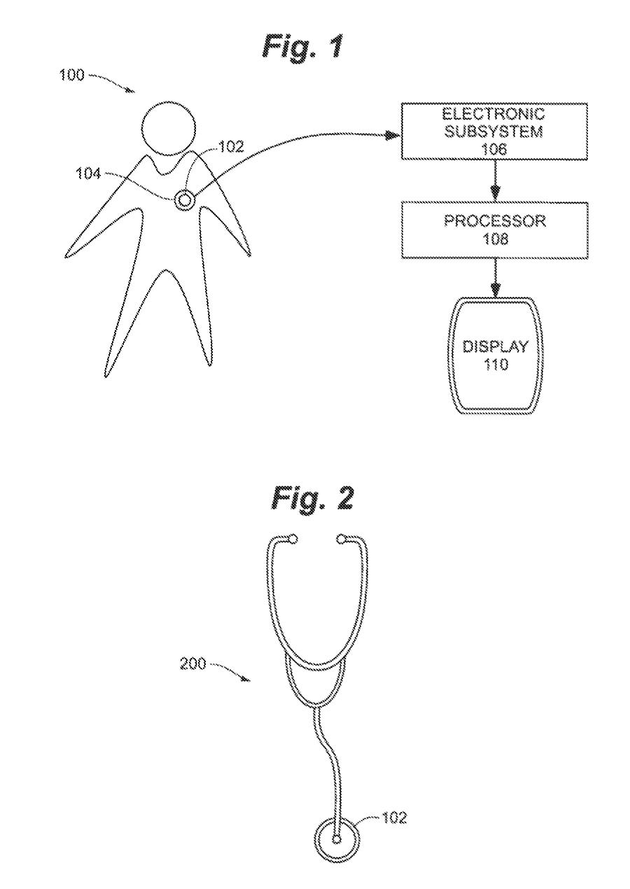

Referring to FIG. 1, an embodiment of a system 100 for detecting cardiovascular disease in a patient includes an acoustic sensor 102 that can be positioned externally on the patient and that receives acoustic data resulting from the cardiovascular system of the patient; a sensor housing 104, in or on which sensor 102 is mounted; an electronic subsystem 106 that conditions the acoustic data received from sensor 102; and a processor 108 that analyzes the conditioned acoustic data to determine presence of cardiovascular disease. The acoustic data can result from blood flow in and/or vibrations propagated along a coronary artery or any suitable blood vessel or physiological structure. The acoustic data can additionally and/or alternatively result from cardiac rhythms or sounds. In some embodiments, system 100 can further include a display or other communication device 110 and/or storage module. Embodiments of system 100 can be used by a physician or other medical professional to determine the presence and/or severity of stenosis in at least one coronary artery of a patient but can additionally and/or alternatively be used by any suitable person to determine and/or predict any suitable cardiovascular-related disease, such as stenosis in any suitable blood vessel, stroke, and hypertension, embodiments of which are discussed herein below.

Embodiments of acoustic sensor 102 function to translate sound waves created within the cardiovascular system of the patient into electrical signals. The electrical signals can be reflective of the nature of blood flow in a blood vessel, and/or the nature of cardiac function (such as changes in the compliance of coronary arteries or changes in vascularization of heart valves). Acoustic sensor 102 can receive acoustic data from at least one of the coronary arteries in embodiments, including: the left anterior descending coronary artery, the right coronary artery, the left main artery, the left circumflex artery, and any of their diagonals, branches, and corollaries. Acoustic sensor 102 can additionally and/or alternatively receive acoustic data from at least one of the following: the carotid artery (such as to detect stenosis as a predictor factor for stroke and/or systemic atherosclerosis), a renal artery (such as to detect stenosis as a predictor factor for a kidney transplant rejection), cardiac rhythms (such as to detect cardiac gallop rhythm S3 or S4 sounds, Dock's murmur, mitral or tricuspid valve papillary ischemia, and/or any suitable cardiac conditions) or any suitable blood vessel or location on the patient (such as to detect ischemia, stenosis, hypertension or other cardiovascular diseases). As such, acoustic sensor 102, in embodiments, can be used in the detection of coronary artery disease, thrombosis development, aortic aneurysm, valve abnormalities, papillary muscle dysfunction, S3 and/or S4 indicators of heart dysfunction, and vessel turbulence; and in screening for sudden cardiac arrest and pulmonary hypertension, among others.

In use, acoustic sensor 102 and sensor housing 104 can be placed externally on a patient and positioned near a blood vessel and/or area of the heart where acoustic data is to be obtained and analyzed. Acoustic sensor 102 can be positioned on the chest of the patient, such as on the fourth left intercostal space of the patient, to obtain acoustic data for the coronary artery, but can additionally and/or alternatively be positioned in any suitable location to obtain acoustic data for other arteries.

Acoustic sensor 102 can receive multiple sets of acoustic data from multiple sources, which may be used in combination for greater device sensitivity. In some embodiments, acoustic sensor 102 can be repositioned in various locations to receive acoustic data from a combination of multiple locations. In some embodiments, system 100 can include a plurality of acoustic sensors that receive acoustic data from any suitable combination of locations. As an example, system 100 can include four sensors that receive acoustic data from each coronary artery. As another example, system 100 can include two sensors that receive acoustic data from each of the left anterior descending coronary artery and the right coronary artery. As another example, system 100 can include two sensors that receive acoustic data from the carotid artery and a coronary artery.

In an embodiment, acoustic sensor 102 comprises piezoelectric material but can alternatively be a mechanical or acoustic wave sensor, microphone, hydrophone, sonar sensor or any suitable acoustic, ultrasound and/or vibration transducer or sensor. The term “acoustic sensor” generally will be used herein throughout for convenience, but use of this term is not meant to be limiting with respect to the type, configuration or characteristics of the sensor, with examples relevant to particular embodiments given, if applicable. For example, sensor 102 can collect at least one of acoustic, seismic, compliance, pressure, flow and/or velocity data inside or outside vessels to determine a disease state of that or an associated vessel. Acoustic sensor 102 can also, in embodiments, be used with an impedance matching material, such as a gel or other fluid similar to those used during ultrasound examinations.

Acoustic sensor 102 can also be used in combination with signal conditioning, filtering, amplification, translation, scaling and/or noise reduction or cancelation circuitry, at least some of which will be discussed in more detail herein below. Such circuitry can be integral with acoustic sensor 102, with sensor housing 104 and/or with some other component of system 100. For example, an embodiment comprises a vibration transducer as acoustic sensor 102, an amplifier, a speaker or speaker jack and digital filtering circuitry to establish at least one impulse transfer function corresponding to vascular changes associated with turbulence, tissue compliance changes, vessel calcification and/or plaque development. In the signal path before filtering, a pre-emphasis of high frequencies in dependence on the thickness of tissue present between an actual sound source and the transducer can be performed. Digital filtering circuitry can also be used in embodiments to provide de-emphasis which establishes at least one impulse transfer function as previously mentioned. Other embodiments can comprise digital pattern recognition circuitry and/or algorithms for windowing an acoustic signal to adaptively remove noise from the surroundings and suppress repetitive signals in an observed signal.

Embodiments of sensor housing 104 function to provide a structural support for acoustic sensor 102 and to provide an interface for positioning acoustic sensor 102 on the patient. Sensor housing 104 can partially or fully encase acoustic sensor 102 and can include a power source such as a battery, or connections from a power source, to acoustic sensor 102.

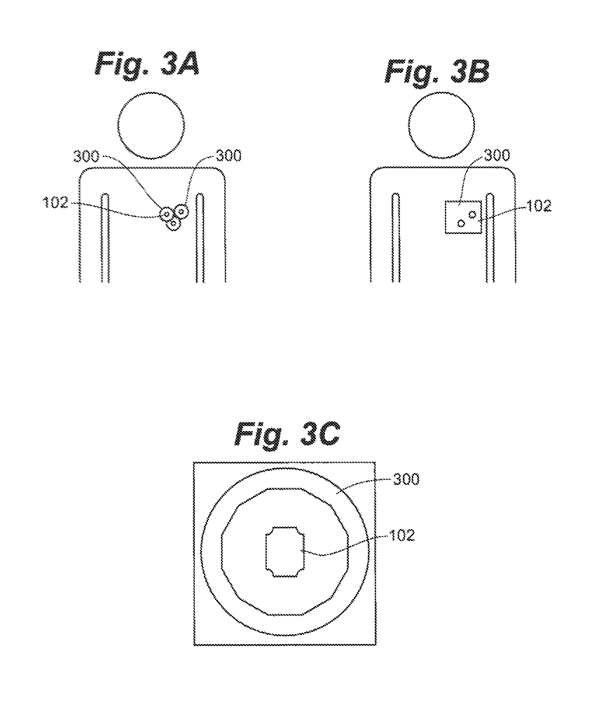

The particular configuration of sensor housing 104 can vary in embodiments. For example, FIG. 2 depicts sensor housing 104 as a stethoscope-like device 200. An embodiment of stethoscope-like device 200 includes a distal end in or on which acoustic sensor 102 is attached. The distal end is hand-held in an embodiment, as is customary for a stethoscope device, and can be positioned by a user to place acoustic sensor 102 in a suitable location, such as the fourth left intercostal space of the patient. Stethoscope-like device 200 does not provide the user with audible sound associated with the acoustic data gathered by the acoustic sensor in an embodiment, though in other embodiments audible or visual feedback can be provided. For example, in some embodiments stethoscope-like device 200 can include an earpiece worn by the user, or any suitable audio interface, that provides the user with audible sound associated with the acoustic data, such as processed or highlighted data, and/or any suitable audio associated with the determination of the presence of cardiovascular disease.

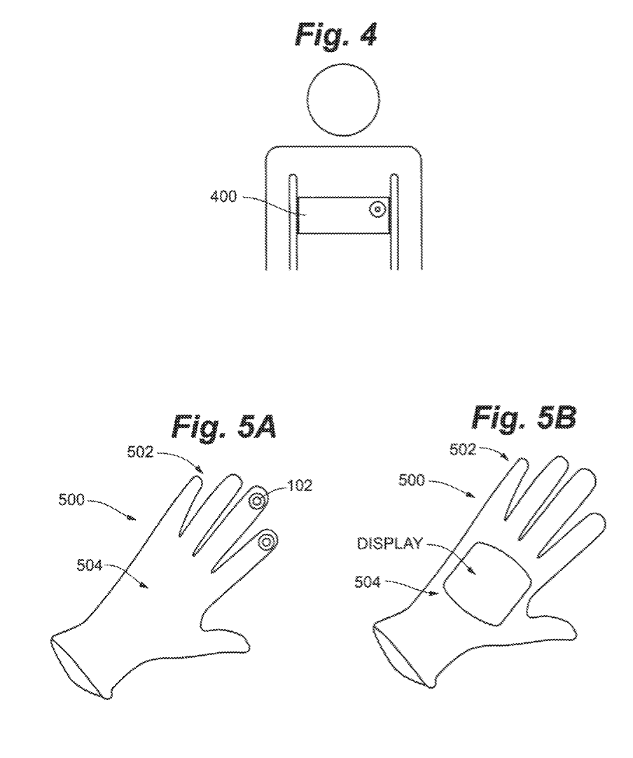

As depicted in various embodiments in FIG. 3, sensor housing 104 comprises a dermal patch 300. Dermal patch 300 is an adhesive patch that removably mounts onto the skin of the patient in embodiments and includes an underside surface or other suitable surface to which acoustic sensor 102 is attached. Acoustic sensor 102 can be attached to dermal patch 300 with an adhesive such as glue, sewn onto the dermal patch, or in any suitable manner. Embodiments of dermal patch 300 can comprise one or more acoustic sensors 102.

For example, in the embodiment depicted in FIG. 3A, dermal patch 300 can include one acoustic sensor such that to receive acoustic data for multiple locations, multiple dermal patches are arranged relative to one another on the patient. FIG. 3A depicts three dermal patches 300, though more or fewer can be used in other embodiments. Alternatively, as depicted in FIG. 3B, dermal patch 300 can include one or more acoustic sensors 102 prearranged into approximate relative positions to gather acoustic data from coronary arteries or any suitable group of locations. Dermal patch 300 can be selected from a group of available sizes of dermal patches, to accommodate patients of multiple sizes and/or shapes and/or to be configured to conveniently and comfortably adhere to one or more areas of the body. Dermal patch 300 can be made of a biocompatible cloth, plastic, or other suitable material and adheres to the patient with a biocompatible adhesive in embodiments.

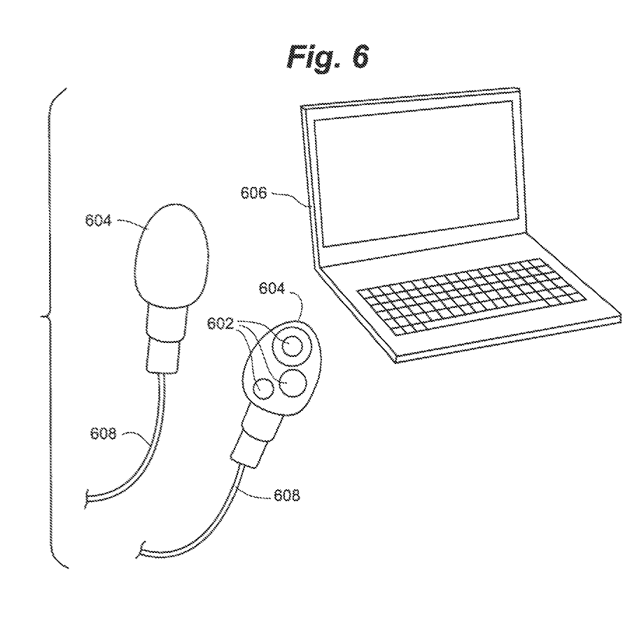

As depicted in FIG. 4, sensor housing 104 comprises a body wrap 400 that can wrap around the chest, abdomen, torso, shoulder, neck or any suitable portion of the patient. Body wrap 400 can be similar to the dermal patch (300) variation, except that body wrap 400 can include elastic to securely conform to the patient. Embodiments of body wrap 400 can be a strip of material wound around the body and fastened with Velcro, snaps or some other means, a band of material pulled onto the body, a shirt, or any suitable garment that wraps around the body or a portion thereof of the patient.

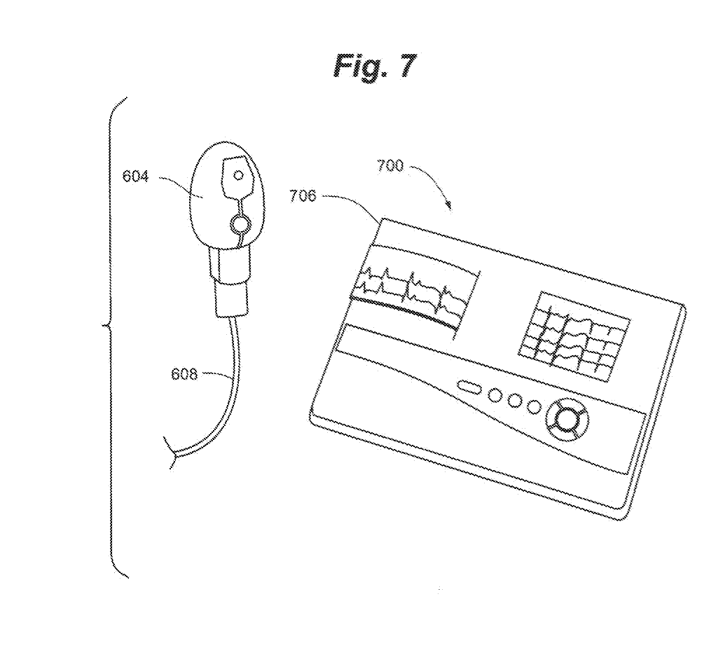

FIG. 5 depicts another embodiment in which sensor housing 104 comprises a glove 500 worn on a hand of the user. Glove 500 can include a finger portion 502 that covers the fingers of the user and/or a palm portion 504 that covers the palm of the user. In another embodiment, finger portion comprises a unitary portion such that glove 500 comprises a mitten-like structure, with or without a thumb portion. One or more acoustic sensors 102 are located on finger portion 502 and/or palm portion 504 of glove 500 in embodiments, such that the user can position and press their fingers and/or palm onto or near the patient to receive acoustic data. In other embodiments, acoustic sensor 102 can be located in any suitable location on glove 500.

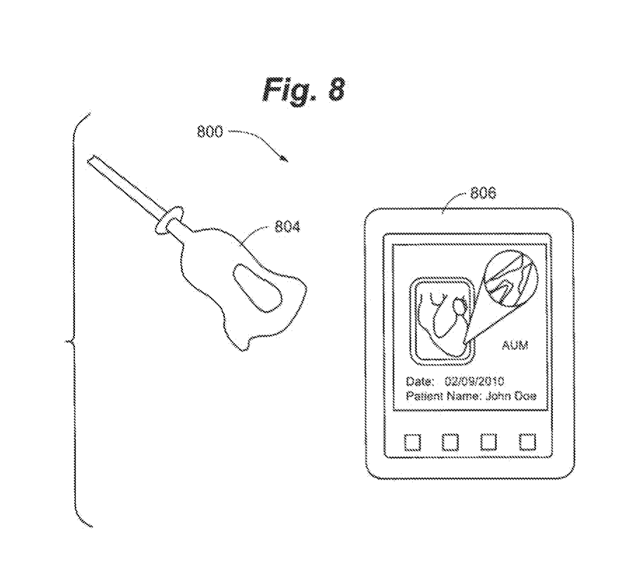

In another embodiment, sensor housing 104 can comprise a catheter, probe and/or lead. One or more acoustic sensors 102 can be placed in or on the catheter or lead for assessing one or more vessels for stenosis, thrombus or plaque development either in real-time or as an implanted monitoring system. Embodiments of such a system can be implanted subcutaneously, under the muscle, inside of an on-board device, as a single sensor and/or as a sensor outside of the body. Such an embodiment, similar to the embodiments discussed below with respect to FIGS. 6-9, can be used by a medical professional with one or more of a hand-held device, personal digital assistant (PDA), smart phone, microcontroller, remote monitoring system, computer, tablet and/or other system for feedback, processing and/or diagnostic purposes. Embodiments can also be used in an automobile or other equipment as an alert of a heart attack in a driver, operator or passenger to an on-board processor for alert to either those in the vehicle or to a remote monitor.

FIGS. 6-9 depict various further embodiments in which sensor housing 104 generally comprises a hand-held device. The particular configuration and features of the hand-held device can vary according to an intended use, environment and/or user. For example, some embodiments can be configured for clinical environments, such as hospitals, clinics, offices of medical professionals and the like, while other embodiments can include convenience features making them suitable for field use, such as in ambulances and other transport vehicles, emergency departments and First Responder kits, military and/or temporary field medical facilities, rudimentary clinical environments, developing or disaster areas, and the like. Still other embodiments can be customized such that they can be used by non-medical professionals for quick initial feedback, as will be discussed in more detail below.

FIG. 6 depicts a hand-held sensor housing 604 configured for use with a notebook or other portable computer device 606 and can be suitable, for example, for use in ambulances and other transport vehicles, emergency departments and First Responder kits, among others. In an embodiment, sensor housing 604 includes a plurality of acoustic sensors 602 configured to be placed externally on a patient. Sensor housing 604 can be wired to prevent loss or misplacement and can communicate with computer device 606 via a USB cable 608 or some other suitable wired communication technique. In another embodiment, sensor device 604 and computer device 606 communicate wirelessly, such as via radio frequency (RF), BLUETOOTH or some other suitable communication technique or protocol. Hand-held sensor housing 604 can be grasped and held by the user, and is pressed against or passed near the coronary artery or other location to receive acoustic data. Hand-held sensor housing 604 can conform to the grasp of a hand of the user in embodiments, such as by having a bulbous shape and/or finger grips, or may be rectangular or any suitable shape. The particular shape and configuration of sensor housing 604 in FIG. 6 is but one, non-limiting example.

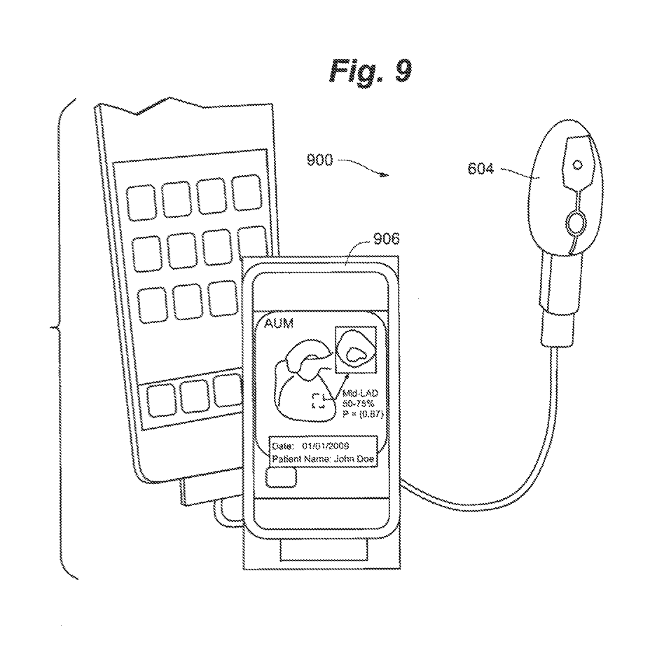

FIG. 7 depicts an embodiment of a combined acoustic sensor/EKG system 700. System 700 utilizes acoustic or ultrasound data from acoustic sensor 102 (not visible in FIG. 7) in hand-held sensor housing 604 in concert with an electrical signal of an electrocardiography (EKG) system 706 for clinical diagnosis and therapy. System 700 can be used, for example, by surgeons, anesthesiologists and other medical professionals for non-cardiac pre-surgical screening, such as in conjunction with conventional and accepted pre-surgical risk assessment and scoring and/or to optimize medical therapies including beta blockers or statins during surgery. System 700 can be used in particular to determine the presence of ST elevations. In embodiments, sensor housing 604 and acoustic sensor 102 are generally compatible with any make or model of EKG system 706. Sensor housing 604 is depicted as wired in the embodiment of FIG. 7 but can be wireless in other embodiments, such as is discussed herein with respect to other embodiments. As depicted, EKG system 706 comprises a display for presenting results. In other embodiments, a display can be integral with one or both of housing 604 and EKG system 706, or a separate display device can be provided.

In other embodiments, echocardiograms, computed tomography scans, IVUS, fractional flow reserve (FFR), CCTA, angiographic studies, cardiac MRI, nuclear scans, calcium scores and/or stress EKGs can also be used with acoustic sensor 102 and hand-held sensor housing 604. Embodiments can also be used with a cardiac defibrillator or pacemaker to determine the presence of compliance changes in the heart, flow-limiting lesions, myocardial infarction or thrombus. Thus, embodiments can utilize either or both of an implantable or external device. Still other embodiments can be used with other technologies, techniques and therapies, such as using acoustic sensor 102 and its data for one or more of optimizing medical therapy; determining flow-limiting, clinically relevant lesions; determining sub-clinical lesions; determining intervention staggering, i.e., performing multiple stent operations and determining which is the most emergent issue; and/or determining where to localize OCT, IVUS and FFR measurements. Still other embodiments can utilize imaging techniques, such as infrared, temperature, ultrasound imaging, Doppler ultrasound 2D, X-ray, CT scan, nuclear scan, seismic and/or other subcutaneous imaging techniques, in conjunction to identify the location of a vessel for navigation of sensor housing 604. Yet another embodiment can combine acoustic sensor 102 on-board with a pacemaker or defibrillator as an alert mechanism for the development or progression of coronary artery disease, stenosis, thrombus and/or congestive heart failure.

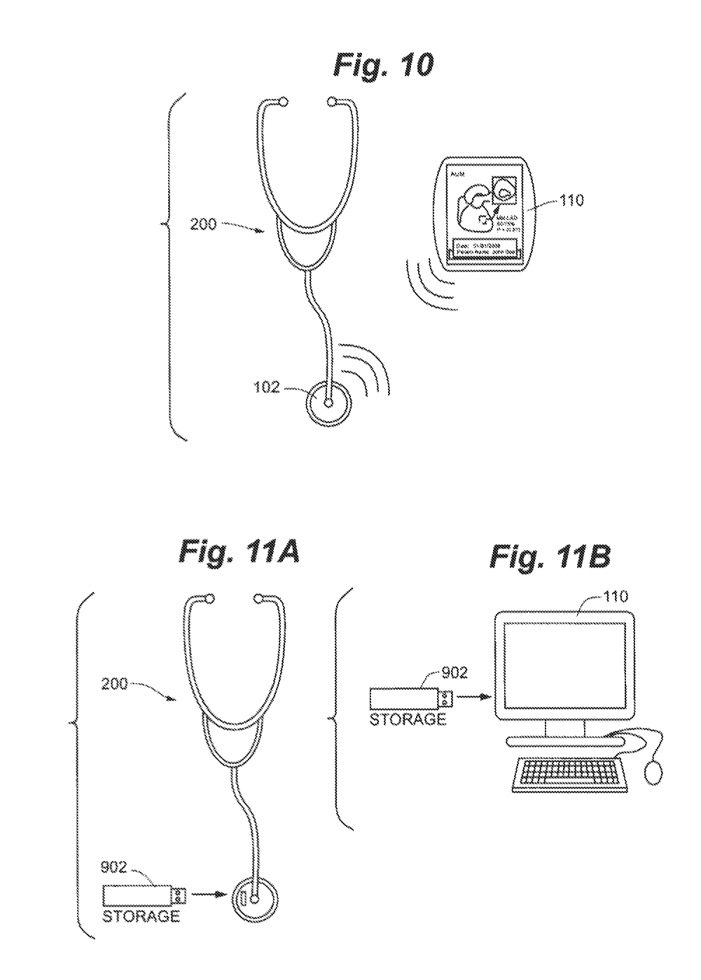

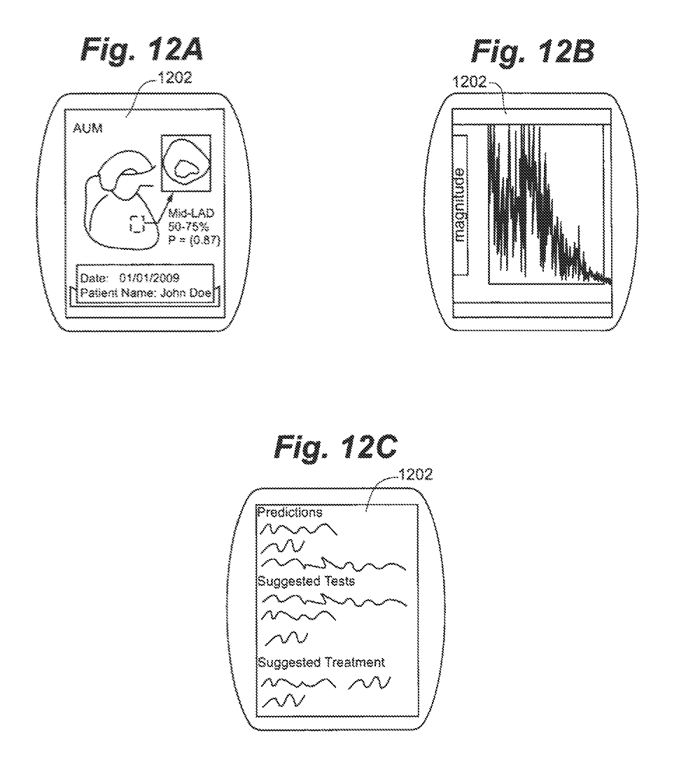

FIG. 8 depicts an embodiment of a system 800 for assessing coronary, carotid and/or renal artery stenosis. System 800 comprises a hand-held sensor housing 804 comprising one or more sensors 102 (not visible in FIG. 8) in communication with a portable computing device 806. Portable computing device 806 can comprise an IPAD, IPHONE, IPOD, personal digital assistant (PDA), smart phone, laptop, notebook, tablet or other computing device in embodiments. In other embodiments, hand-held sensor housing 804 comprises an on-board processor, which can eliminate portable computing device 806. Sensor housing 804 and computing device 806 can communicate wired, such as is depicted, or wirelessly, as is discussed herein with respect to additional embodiments. System 800 can be used, for example, to determine flow limiting lesion locations, with or without fractional flow reserve (FFR), and can assist in the optimization of therapies by using ultrasound techniques of scanning the entire artery. Embodiments of system 800 can thereby provide quantitative information. FIG. 9 depicts an embodiment in which hand-held sensor housing 604 can be used with a smart phone or PDA device 906. System 900 can therefore be highly portable and usable in a variety of environments. Sensor housing 604 and device 906 can communicate wired or wirelessly in embodiments. In one embodiment, data, such as graphical data, is sent from sensor housing 604 to device 906 and can be used for an immediate medical referral. In another embodiment, the data can be sent to a reading center, heart specialist or processor for further processing and/or analysis. While example embodiments of user interfaces, such as for device 906, will be discussed in more detail herein below, device 906 can be suited for a simplified graphical user interface (GUI), such as one which presents a simple YES/NO in response to a test, with optional additional information available. This feature, as well as the portability and relatively affordable price when compared with other analytic and diagnostic systems, can provide an easy-to-understand output and make the embodiment of FIG. 9 broadly suitable for a variety of users and situations.

Another embodiment can substitute patch 300 for housing 604 in the system of FIG. 9. Such an embodiment can be a single-use, consumer-level system that provides a simple output directing the consumer for further diagnostics and/or to appropriate resources as a result of the use of the system. For example, a consumer can purchase a kit comprising a single-use patch 300 and a card with a code to download a smart phone or PDA application or “app,” such as from ITUNES or a similar source. Patch 300 can communicate with the smart phone wirelessly or wired in embodiments. The app, once downloaded and installed on the consumer's smart phone, can direct the consumer in applying the patch and then run the test. If the results warrant further medical attention, the app can provide such output. The app can also provide lifestyle recommendations, such as diet and exercise information, as well as information regarding heart attack warning signs to further educate the consumer.

The sensor housing can be one of the aforementioned embodiments, or the sensor housing can be any combination of these variations, or any suitable housing supporting acoustic sensor 102 in any suitable manner. For example, the embodiments of FIGS. 3A and 5 can be combined such that the sensor housing is a glove including multiple dermal patches that may be arranged relative to one another on the glove. Other possibilities also exist, as appreciated by those skilled in the art.

Referring again to FIG. 1, electronic subsystem 106 of embodiments functions to condition the acoustic data into a format more appropriate for analysis. The electronic subsystem may be directly or indirectly coupled to acoustic sensor 102 such as through a cable, BLUETOOTH, RF or the internet. Electronic subsystem 106 can include electronic elements that perform signal processing functions such as amplification, filtering, downsampling, analog-to-digital signal conversion, high/low frequency band translation and/or noise cancellation. For example, electronic subsystem 106 can apply a series of filters to eliminate non-stenosis frequencies from the data and frequencies above approximately 100 Hz, such as the electronic subsystem described in commonly owned U.S. Pat. No. 7,520,860, which is incorporated herein by reference in its entirety. Other embodiments of electronic subsystem 106 can include any suitable elements to perform any suitable signal processing of the acoustic data. For example, the circuitry previously mentioned for use in conjunction with acoustic sensor 102 can be incorporated with or into electronic subsystem 106.

Processor 108 of embodiments functions to analyze the conditioned acoustic data to determine the presence of cardiovascular disease and/or to make another suitable conclusion. Processor 108 can be directly or indirectly coupled to the electronic subsystem and/or acoustic sensor such as through a cable, BLUETOOTH, RF or the internet. Processor 108 can calculate a fast Fourier transform (FFT) analysis of the acoustic data, and uses the FFT data to diagnose coronary artery stenosis, or any suitable cardiovascular or other disease. Depending upon where the acoustic data was collected with respect to a patient, the processor can determine stenosis in one or more of the left anterior descending coronary artery, the right coronary artery, the left main coronary artery, and the left circumflex artery, among others. In particular, the processor can plot the FFT data on a diagnostic graph, which is a log-log plot of the frequency spectrum in an embodiment, with harmonic magnitudes of the FFT data on the y-axis and the frequency of the harmonic on the x-axis, as described in U.S. Pat. No. 7,520,860, as referenced above. The diagnostic graph can be a physical plot and/or can be a computational comparison between each intended axis of the plot. Processor 108 can analyze the diagnostic plot, first determining whether the frequency spectrum defines a bell-shaped curve, and then comparing the characteristics of the bell-shaped curve to an upslope threshold, a downslope threshold, and a maximum magnitude threshold. For example, the processor determines whether the upslope of the curve (the lower frequency where the slope of the curve rises) occurs substantially at or closely above 50 Hz, whether the downslope of the curve (the higher frequency where the slope of the curve lowers) occurs substantially at or below 80 Hz, and also whether the maximum harmonic magnitude of the FFT data is above 2.5 units. If processor 108 determines that the plotted FFT data meets all four criteria, then processor 108 determines occlusion of the artery. Processor 108 can additionally and/or alternatively calculate the sum of the energy under the bell curve and analyze the sum to determine an estimate of the percentage of occlusion of the artery. For example, depending upon the magnitude of the sum of the energy under the bell curve, the artery may be determined to have 0-25%, 25-50%, 50-75%, 75-90%, or more than 90% occlusion of the artery. Similarly, the artery may also be determined to have more than 75% or less than 75% occlusion. Other thresholds and/or other curve characteristics may be used to determine other kinds of cardiovascular disease.

Processor 108 can additionally and/or alternatively analyze the acoustic data of one coronary artery to predict occlusion in at least one of the other coronary arteries. The processor can also additionally and/or alternatively analyze the acoustic data of two or three coronary arteries to predict overall systemic cardiovascular disease. These predictions can, for example, involve consideration of the percentage of occlusion of the analyzed artery or arteries, and/or the proximity of one coronary artery to another.

Processor 108 can additionally and/or alternatively analyze the acoustic data to determine whether an occlusion of a coronary artery is clinically relevant. For example, processor 108 can suggest consideration of further diagnostic tests, such as deliverance of a stress test, echocardiogram, and/or calcium scoring. Furthermore, processor 108 can suggest consideration of intervention and/or treatment, such as a coronary artery bypass, a coronary artery angiography or angioplasty, pharmaceutical treatment, lifestyle modification, smoking cessation, and/or weight management.

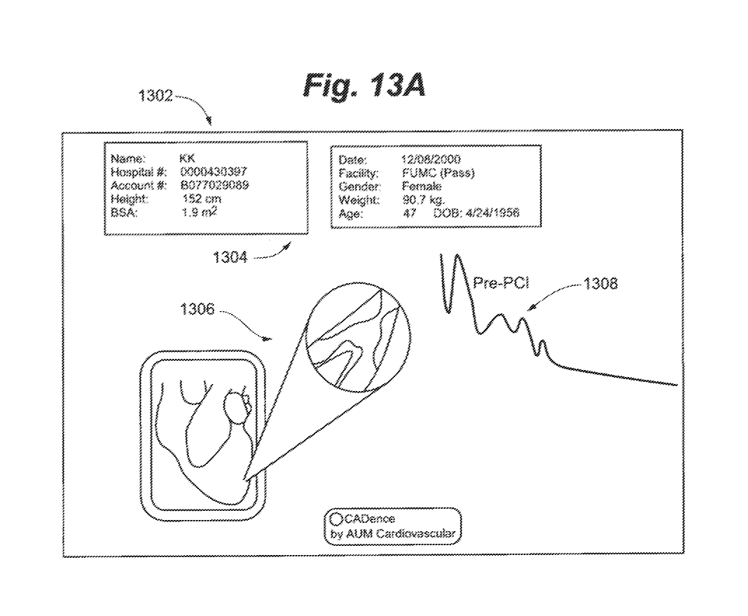

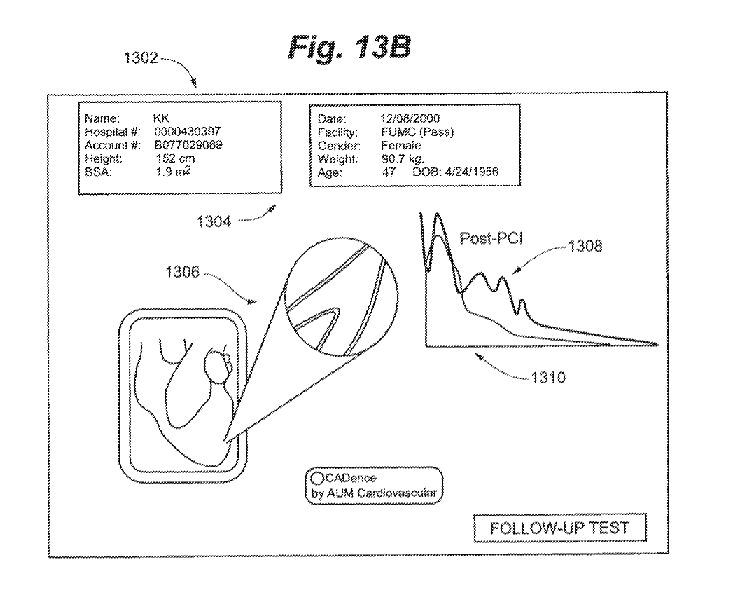

As shown in FIGS. 6-9, embodiments of system 100 can further include a communication device or display 110 coupled to processor 108 that functions to present information such as the acoustic data, analysis, and/or conclusions of processor 108 to a user and/or to the patient. For example, and referring to FIG. 5B, a communication device 110 can be directly mounted to sensor housing 500. In a second variation, such as is depicted in FIGS. 6-9, communication device 110 can be remotely connected to sensor housing through a wired connection (such as a USB cable, as shown in FIG. 6), a wireless connection (such as BLUETOOTH, as shown in FIG. 10), or a portable memory (such as a flash drive 902 for exchanging data between sensor housing 200 and communication device 110, as shown in FIG. 11), a wired or wireless network connection (such as an internet connection, not shown), or any other suitable device or method. Communication device 110 can be an integral part of the system or can be a separate stand-alone device, such as a personal digital assistant (PDA), a cell phone, an electronic book, a pager, a personal computer such as a laptop, desktop or tablet computer, an electronic health record, a customized device, or any suitable display.

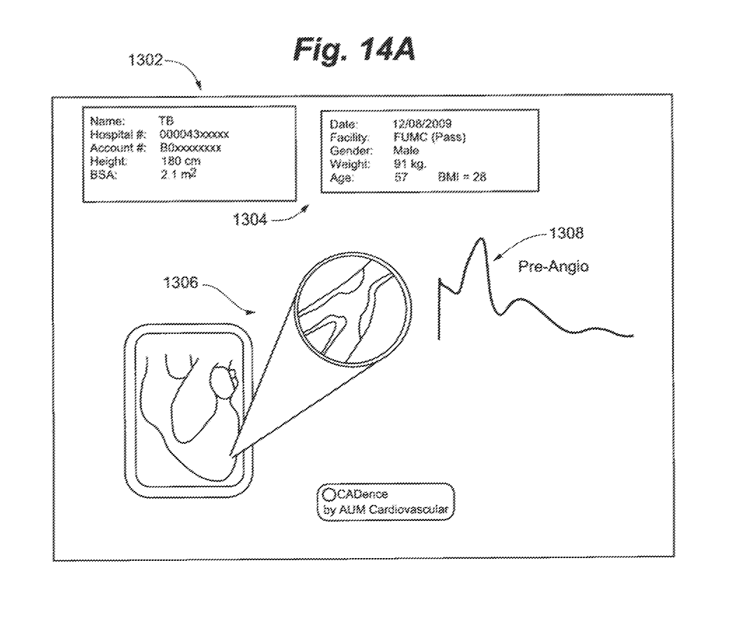

Communication device 110 includes a visual display in embodiments. The visual display can present a graphical representation of data, the conclusion, and/or any suitable information to a user via a graphical user interface (GUI). As shown in FIG. 12A, an embodiment of a visual display 1202 presents a realistic or sketch drawing graphical depiction of the coronary artery or arteries. The graphical depiction can include a full or partial view of the heart with highlighted regions of disease. The graphical depiction can additionally and/or alternatively include a cross-sectional view of one or more coronary arteries illustrated with percent occlusion represented by fatty blockages, a pie chart, and/or numerical labels. As shown in FIG. 12B, another embodiment, or another screen, of visual display 1202 presents the diagnostic graph and can illustrate the bell curve upslope threshold and/or downslope threshold with lines on the plot, such as to demonstrate the algorithm for determination of artery stenosis. Such a display can be most suited for medical professionals analyzing or desiring more information regarding an outcome. As shown in the embodiment of FIG. 12C, visual display 1202 presents predictions as well as the suggestions of the processor to consider additional diagnostic tests, intervention, and/or treatment. Other embodiments of visual display 1202, however, can be any combination of the above or other variations, and include any suitable images and/or text for conveying any suitable information.

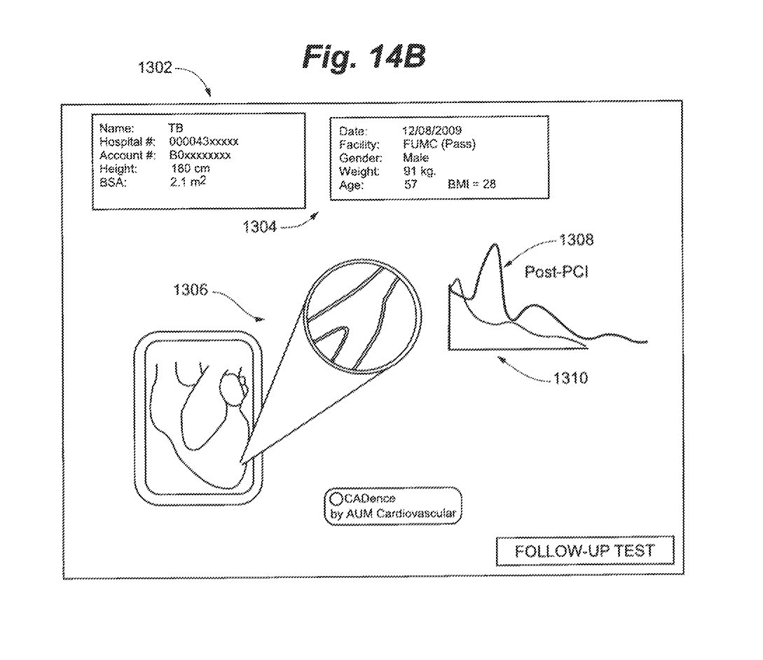

For example, FIG. 13 depicts an embodiment of a graphical user interface (GUI) 1302. GUI 1302 can be displayed on visual display 1202 in conjunction with any of the embodiments discussed herein above, such as those depicted in FIGS. 6-9. GUI 1302 can include basic patient identifying and statistical information 1304. Embodiments of GUI 1302 can also include a cross-sectional view 1306 of one or more coronary arteries illustrated with percent occlusion represented by fatty blockages. In the example embodiment of FIG. 13, the particular patient has an approximate 90% blockage of the proximal left anterior descending coronary artery. Therefore, view 1306 shows an artery having blockage. GUI 1302 also includes an output waveform 1308 from the patient's scan using acoustic sensor 102. Waveform 1308 is indicative of coronary artery disease.

FIG. 13 is based on actual patient data. Following the scan illustrated in GUI 1302 of FIG. 13A, the patient underwent a percutaneous coronary intervention (PCI) or angiogram. FIG. 13B depicts GUI 1302 with an updated view 1306 as well as a new waveform 1310 taken from a scan post-PCI and presented superimposed with pre-PCI waveform 1308. As can be seen by comparing FIGS. 13A and 13B, a significant change in waveform 1308/1310 occurred, illustrated by the new shaded waveform post-PCI at 1310 and demonstrating the effectiveness of embodiments of the system in detecting coronary artery disease by acoustic data, visible in waveform 1308.

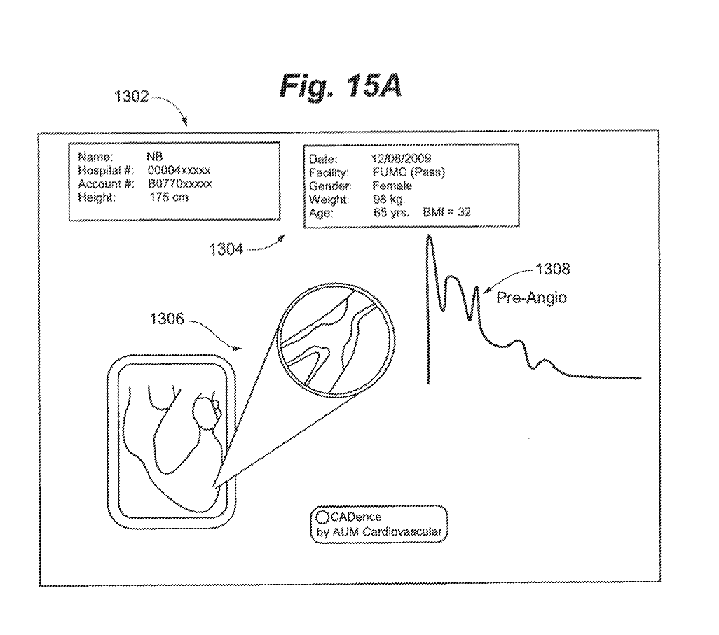

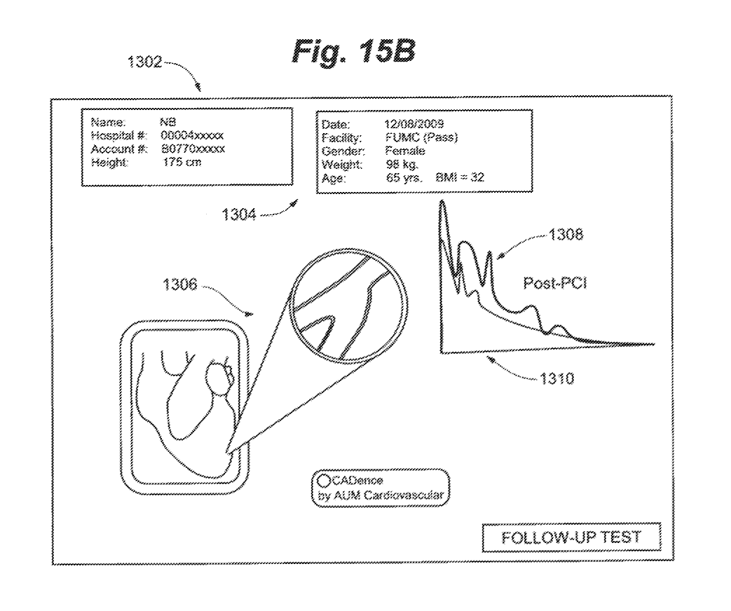

Two additional examples are presented for two other patients in FIGS. 14 and 15, with pre-PCI or angiogram results presented in FIGS. 14A and 15A and post-PCI or angiogram results presented in FIGS. 14B and 15B. Patient 2 (FIG. 14) had an 80% blockage of the left anterior descending coronary artery, and patient 3 (FIG. 15) had a 60% blockage of the left anterior descending coronary artery with a 75-90% blockage of diagonal branch D2.

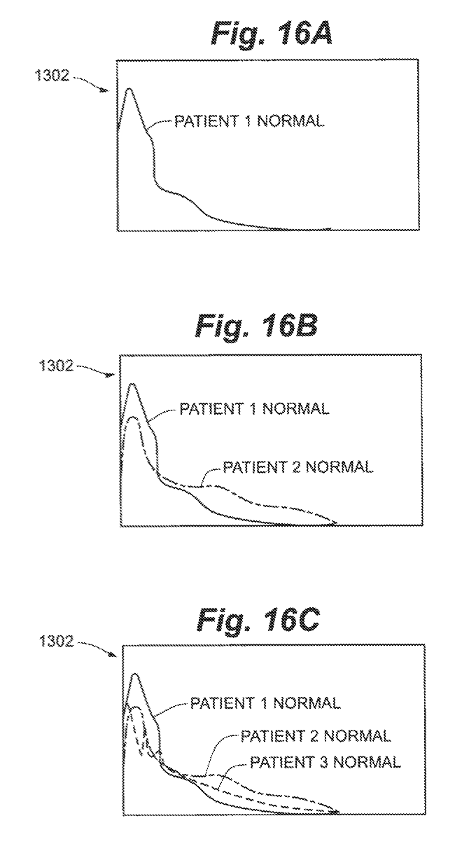

FIG. 16 is another embodiment of GUI 1302. FIG. 16A depicts normal waveform 1310 for patient 1 (FIG. 13), FIG. 16B adds normal waveform 1310 for patient 2 (FIG. 14) and FIG. 16C adds normal waveform 1310 for patient 3 (FIG. 15). FIG. 16D then adds the waveforms associated with each patient pre-scan, showing the 90% blockage for patient 1, 80% blockage for patient 2 and 60% blockage for patient 3 superimposed with the normal scans for each patient. FIG. 16 therefore illustrates the effectiveness of embodiments in detecting coronary artery disease while presenting the results in clear, easy to recognize visual outputs.

Referring generally to FIGS. 13-16, waveforms 1310 are considered to be “normal” waveforms for the patient as compared with waveforms 1308. A “normal” waveform can be defined in embodiments, with frequency ranges or bands associated with “abnormal” chosen. For example, a variety of peaks within a range of about 30 Hz to about 70 Hz is associated with S3, S4, cardiomyopathy, MI, ventricle compliance changes and/or the longitudinal/transverse waves associated with velocity or turbulent flow changes in a diseased coronary artery. Refer, for example, FIG. 13 for patient 1, which shows peaks at approximately 40, 45, 50 and 60 Hz and FIG. 14 for patient 2, which shows a peak at approximately 55 Hz. A variety of peaks within a range of about 250 Hz to about 1,000 Hz is associated with turbulence or velocity changes in the coronary artery, such as because of a partial blockage, MI, cardiomyopathy and/or ventricle compliance. Refer, for example, to FIG. 13 for patient 1, which shows a peak at about 650 Hz and FIG. 14 for patient 2, which shows peaks at approximately 350, 600 and 750 Hz.

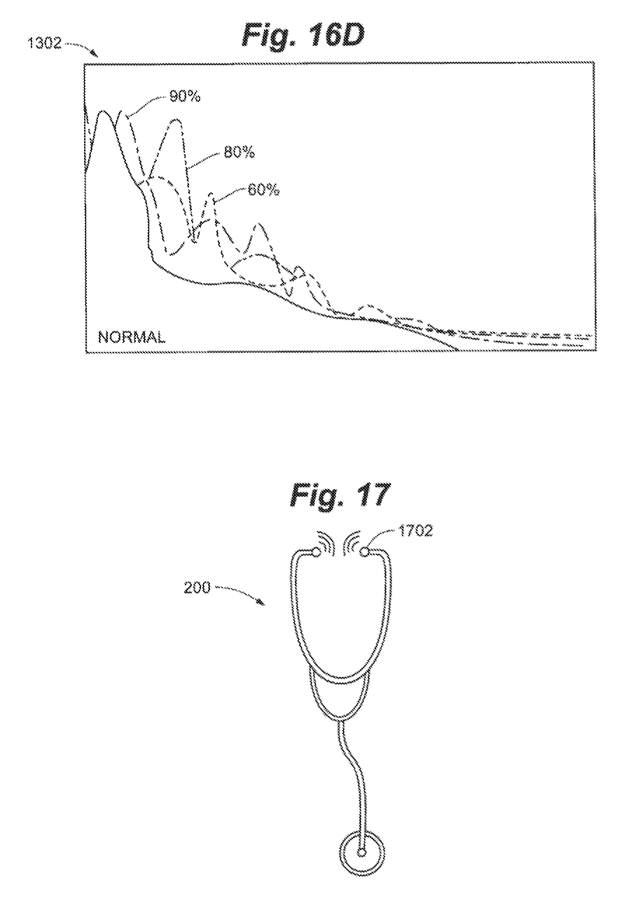

Referring again to FIG. 1 as well as FIG. 17, communication device 110 can additionally and/or alternatively include an audio speaker. The audio speaker can convey information similar to that of the visual display, but in an audio format that can be heard, which can be more convenient in particular environments and settings and/or preferred by certain users and medical professionals. The audio speaker can alternatively and/or additionally include the simulated or actual sound of blood flow received by acoustic sensor 102. In some embodiments, the audio speaker can be coupled to the sensor housing or any suitable part of the system, such as the earpieces 1702 of a stethoscope as depicted in FIG. 17.

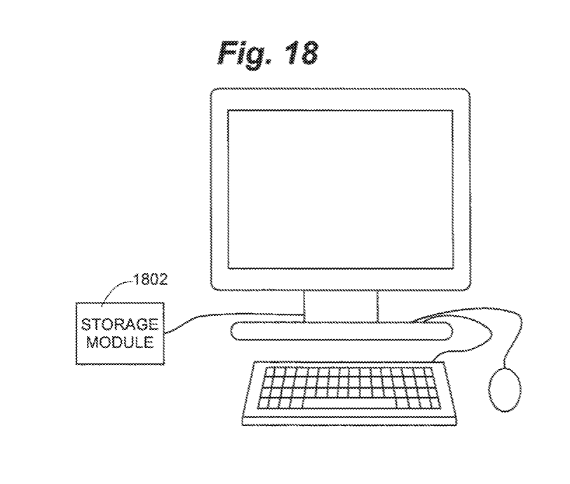

As shown in FIG. 18, embodiments can further include a storage module 1802 that functions to store information including the acoustic data, acoustic data analysis, the conclusions of processor 108, and/or graphical representations. Storage module 1802 can be a local or remote storage device, such as a computer hard drive, flash memory, or a server. Storage module 1802 can store any of the information at any particular time. For example, storage module 1802 can store the acoustic data after acoustic sensor 102 receives the acoustic data, and storage module 1802 can be used to transfer the acoustic data to electronic subsystem 106 for conditioning, to processor 108 for analysis, and/or to communication device 110 for presentation of information. In another example, storage module 1802 stores information after processor 108 analyzes the acoustic data, for purposes such as for electronic medical records.

Although omitted for conciseness, embodiments can include every combination and permutation of the various sensors, sensor housings, communication devices, and storage modules.

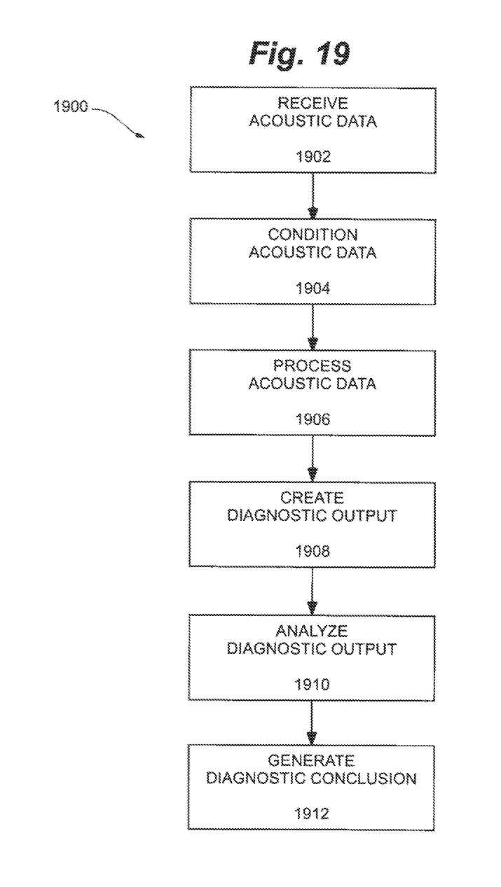

Referring to FIG. 19, a flowchart of an embodiment of a method 1900 for detecting cardiovascular disease is depicted. At 1902, acoustic data is received. The acoustic data can be received from at least one of the coronary arteries in an embodiment, and the collection of the acoustic data can be performed by at least one acoustic sensor, embodiments of which are discussed above, or by any suitable sensor. Data can be received solely from the acoustic sensor, or data can additionally be received from other complementary devices; refer, for example, to FIG. 7 and the related discussion. Data collection improvement or enhancement techniques can also be used, such as the administration of adenosine to induce hyperemia in order to further accentuate acoustic components of interest. Additionally or alternatively, a patient can be instructed to lean forward during a scan with the acoustic sensor in embodiments in order to further accentuate acoustic components of interest.

At 1904, the acoustic data is conditioned. Conditioning the acoustic data can include one or more of amplifying the acoustic data, filtering the acoustic data, downsampling the acoustic data, converting the acoustic data from an analog to a digital signal, and/or canceling noise in the acoustic data, and can be performed by the electronic subsystem of the system, embodiments of which are discussed above.

At 1906, the acoustic data is processed. Processing can include calculating a fast Fourier transform of the conditioned data to obtain fast Fourier transform (FFT) data.

At 1908, a diagnostic output is created. Diagnostic output can include plotting the FFT data as magnitude vs. frequency to create a diagnostic graph or presenting the data in some other form. Graphical techniques such as scatter plots, correlations and/or cross-tabulations can also be used to present data either to a person or to another processing unit for further analysis to detect coronary artery disease. Graphical plots can also correspond to Log Hz vs. dB, Log Hz vs. frequency coefficient, smoothed frequency curves and/or spectrograms. Such plots can be utilized, such as at 1910, to find frequency peaks and/or energies of interest to detect coronary artery disease.

At 1910, the diagnostic output is analyzed. The analysis can be computer-driven or user-driven in embodiments or a combination thereof.

At 1912, one or more diagnostic conclusions are generated based on the analysis of the diagnostic graph. Embodiments can be similar to those described in U.S. Pat. No. 7,520,860, as referenced above, except as described below. Analysis can also be carried out according to more or more statistical methods including but not limited to Analysis of Variables (ANOVAI), Correlation, Factor Analysis, and Pearson's Chi-Square test to detect coronary artery disease. Data can be used to determine flow and velocity changes in a vessel associated with intravascular anomalies to including coronary artery disease, renal artery stenosis, carotid artery stenosis and peripheral artery disease. Embodiments can also determine Reynauld's Number for a vessel. Data can also be used as an input to a decision tree based on a multitude of patient risk factor assessment questions for the purpose of assessing potential for development of coronary artery disease, thrombus, intravascular stenosis, heart attack, stroke and/or others.

Embodiments of method 1900 can further include communicating at least one of the acoustic data, the diagnostic graph, and diagnostic conclusion. Communicating can be visual and/or audio and can be performed by the visual display and/or audio speaker of the system described above. Embodiments of method 1900 can also further include storing at least one of the acoustic data, the diagnostic graph, and the diagnostic conclusion and can be performed by the storage module of the system described above.

Embodiments of method 1900 can be used to determine locations of flow-limiting lesions along the body of a vessel by collecting, at 1902, multiple sets of external thorax data along the length of the vessel, including but not limited to one or more of LMAIN, LAD proximal, LAD diagonals, mid-LAD, apical LAD, LCX, marginals, and RCA. That data can then be displayed, at 1908, on a user-interface as a graph, vessel position or number. Multiple sites can be used to specify lesion location along the body of the coronary artery, the sites including but not limited to one or more of the second left intercostal space, across the thorax to the site above the nipple, under the armpit, about 15 cm below the armpit vertically, at the apex and at the subxyphoid (right coronary artery).

Embodiments can also be used in conjunction with one or more of Framingham risk scores, presurgical screens algorithms, or Emergency Acute Myocardial Infarction protocols to assist in triaging at-risk persons for medical or interventional therapy. Referring, for example, to FIG. 7, embodiments also relate to using ultrasound data to separate patients in need of cardiac intervention with NSTEMI EKG reports.

Embodiments therefore relate to a variety of systems, methods and apparatuses which can use acoustic data in the detection of coronary artery disease. Embodiments can enable fast, non-invasive identification of clinically relevant coronary artery disease, which can ultimately save lives. The non-invasive and convenient aspects of embodiments can be used to meet a large, as yet unmet need in a cost-effective and accurate manner. Results can be provided in real-time and with clarity, providing quick and easily understandable results that can shorten the path to intervention for patients, making embodiments suitable for a wide range of environments, purposes, users and patients. These and other advantages can be provided without special technical training or special dyes or drug regimes but with immediate results.

Various embodiments of systems, devices and methods have been described herein. These embodiments are given only by way of example and are not intended to limit the scope of the invention. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

What is claimed is:

1. A medical device comprising:
at least one acoustic sensor;
a housing coupled to the at least one acoustic sensor and configured to position the at least one acoustic sensor to collect acoustic data, the acoustic data including a representation of at least a sound of blood through an artery of a user;
a processor operably coupled to the acoustic sensor and configured to receive the acoustic data and determine a health-related event based at least on the acoustic data; and
a securing mechanism configured to secure the housing to a portion of the user.

2. The medical device of claim 1, wherein securing mechanism is configured to secure the housing to the portion of the user selected from the group consisting of a chest, an abdomen, a torso, a shoulder, a neck, a finger, and an arm.

3. The medical device of claim 1, wherein the health-related event is at least one of a heart attack, coronary artery disease, thrombosis development, aortic aneurysm, valve abnormalities, papillary muscle dysfunction, heart dysfunction, vessel turbulence, sudden cardiac arrest, or pulmonary hypertension.

4. The medical device of claim 1, wherein the securing mechanism comprises a flexible band.

5. The medical device of claim 1, wherein the securing mechanism comprises a strip of material operably coupled at either end with at least one of Velcro, snaps, or a buckle.

6. The medical device of claim 1, further comprising a visual display operably coupled to the housing and configured to visually display at least one of the health-related event determined by the processor or the acoustic data.

7. The medical device of claim 1, further comprising an electronic subsystem configured to condition acoustic data, wherein the processor is further configured to receive the conditioned acoustic data and determine the health-related event based at least in part on a shape of a waveform of the conditioned acoustic data.

8. The medical device of claim 1, further comprising a battery operably coupled to the housing and configured to provide power to the at least one acoustic sensor and the processor.

9. The medical device of claim 1, wherein the at least one acoustic sensor comprises an acoustic sensor remote from the housing.

10. The medical device of claim 1, wherein the processor is further configured to transmit at least one of the acoustic data or health-related event to a remote electronic device comprising one selected from the group consisting of: a smart phone, a personal digital assistant, a tablet computer, an MP3 player, a laptop computer; a notebook computer; a desktop computer; a monitor; and a second medical device.

11. A device for use in the detection of a health-related event, the device comprising:
a main body;
a sensor operably coupled to the main body;
an electronic subsystem housed substantially within the main body and configured to condition data received from the sensor;
a processor coupled to the electronic subsystem and configured to receive the conditioned data and determine a health-related event;
an electronic display operably coupled to the main body and configured to visually display at least one of the health-related event or the conditioned data;
a battery operably coupled to the main body and configured to provide power to the sensor, the electronic subsystem, the processor, and the electronic display; and
a band configured to secure the main body to an arm of a user.

12. The device of claim 11, wherein the health-related event is at least one of a heart attack, coronary artery disease, thrombosis development, aortic aneurysm, valve abnormalities, papillary muscle dysfunction, heart dysfunction, vessel turbulence, sudden cardiac arrest, or pulmonary hypertension.

13. The device of claim 11, wherein the band is configured to flexibly conform to the arm of the user.

14. The device of claim 11, wherein the sensor is selected from the group consisting of an acoustic sensor, a seismic sensor, a compliance sensor, a pressure sensor, a flow sensor, and a velocity sensor.

15. The device of claim 11, wherein the electronic display is further configured to present a graphical user interface that includes at least one waveform associated with the conditioned data.

16. The device of claim 15, wherein the graphical user interface includes a diagnostic conclusion.

17. A system for determining a health-related event, the system comprising:
a device comprising:
at least one sensor,
a housing coupled to the at least one sensor and configured to position the at least one sensor to collect sensor data, the sensor data including a representation of at least a sound of blood through an artery of a user,
a processor operably coupled to the sensor and configured to receive the sensor data, and
a securing mechanism configured to secure the housing to a body of a user; and
a remote electronic device in communication with the processor,
wherein the processor is configured to transmit the sensor data to the remote electronic device, and the remote electronic device is configured to determine a health-related event based at least on the sensor data.

18. The system of claim 17, further comprising a second sensor operably coupled to the processor and configured to collect sensor data at a position on the user distal from the device.

19. The system of claim 17, wherein the device and remote electronic device are respectively configured to communicate wirelessly.

20. The system of claim 17, wherein the remote electronic device comprises one selected from the group consisting of: a smart phone, a personal digital assistant, a tablet computer, an MP3 player, a laptop computer; a notebook computer; a desktop computer; a monitor; and a medical device.
